(19) United States
(12) Patent Application Publication
(10) Pub. No.: US 2004/0265803 A1
(43) Pub. Date: Dec. 30, 2004

(54) Genes and their genetic products pertinent to microsatellite instable (msi+) tumours

(76) Inventors: Magnus von Knebel Doeberitz, Heidelberg (DE); Peer Bork, Heidelberg (DE); Yan Ping Yuan, Mayerhofstr.1 (DE); Johannes Gebert, Dossenheim (DE); Stefan Worner, Heidelberg (DE); Michael Linnebacher, Schiffwiler (DE)

Correspondence Address: Albert P Halluin, Howrey Simon Arnold & White, 301 Ravenswood Avenue, Box No 34, Menlo Park, CA, 94025, US

(21) Appl. No.: 10/332,522

(22) PCT Filed: Jul. 4, 2001
(86) PCT No.: PCT/DE01/02510

(30) Foreign Application Priority Data
Jul. 7, 2000 (DE) 100 32 608.0

(51) Int. Cl. C12Q 1/68; C07H 21/04; C07K 16/30

(52) U.S. Cl. 435/6; 530/388.8; 530/350; 536/23.5

(57) ABSTRACT

The invention relates to genes comprising coding mononucleotide microsatellites (cMNR) or dinucleotide microsatellites (cDNR). The genes can be isolated from MSI+ tumour cells. Said genes differ from corresponding genes from non-MSI+ (tumour) cells by mutations in the cMNR or cDNR and code for gene products including neopeptides. The invention also relates to the use of the genes and their gene products for the prevention, diagnosis and/or therapy of MSI+ tumours.

The present invention relates to genes pertinent to MSI+ tumors and to their gene products. The invention also relates to a method of identifying such genes and to the use of the genes and/or their gene products for the prevention, diagnosis and/or therapy of MSI+ tumors.

Tumor cells accumulate instabilities (mutations) within genes essential for maintaining normal growth and standard differentiation. Two kinds of genetic instability were identified in human tumors: Chromosomal instability (CIN) and microsatellite instability (MSI), the latter being characterized by variations in length of repetitive DNA sequences in diploid tumor cells. There is a wide difference between CIN and MSI+ tumors as regards the type and spectrum of mutated genes, which refers to different pathways of cancerization, which are, however, not mutually exclusive. MSI occurs in about 90% of hereditary non-polypous-colorectal tumors (HNPCC) and in about 15% of sporadic tumors of the large intestine and further organs and is caused by mutation-induced inactivation of different DNA mismatch repair genes. MSI+ tumors have special histopathologic characteristics. MSI+ tumors are also classified using as a rule microsatellites in non-coding regions or intron sequences. However, there is information that microsatellites are also subject to instability in coding gene regions. This might be highly significant for the tumor formation of MSI+ tumors.

The present invention is thus based on the technical problem of providing a product which serves for studying MSI+ tumors on a molecular level and which should the occasion arise is suited for the diagnosis and/or therapy of MSI+ tumors.

According to the invention this technical problem is solved by the subject matters defined in the claims.

The present invention is based on Applicant's insight that genes contained in MSI+ tumor-coding mononucleotide microsatellites (cMNR) often have instabilities (mutations) in their cMNRs. To this end, he identified about 17,000 coding mononucleotide microsatellites (cMNR) and about 2,000 coding dinucleotide microsatellites (cDNR), comprising repeat units with n≧6 or n≧4, by means of computer-algorithm-assisted database analysis. The genetic instability of 15 cMNRs (n≧9) and 4 cDNRs (n≧5) and the expression of the corresponding genes were investigated in 16 MSI+ and 20 non-MSI+ tumors and cell lines, these analyses focusing on long repeat units. The cMNRs and/or cDNRs showed instability (mutation) frequencies covering from 1-100% in MSI+ tumor cells; however, the cMNRs and/or cDNRs were stable in non-MSI+ (tumor) cells. Most cMNR-containing genes (10 of 15=66%) were highly expressed in all of the MSI+ and non-MSI+ (tumor) cells, no significant correlation between expression level and mutation frequency being observable. In addition, he found out that the instable cMNR- and/or cDNR-bearing genes code for neopeptide-comprising gene products and that these gene products are suited for the immunization of an individual against MSI+ tumors and/or the preliminary stages thereof. Reference is made to FIGS. 1-3, Tables 1-3 and the below examples.

The subject matter of the present invention thus relates to genes having coding mononucleotide microsatellites (cMNRs) or dinucleotide microsatellites (cDNRs), wherein the genes can be isolated from MSI+ tumor cells and differ from the corresponding genes from non-MSI+ (tumor) cells by mutations in the cMNRs or cDNRs and code for neopeptide-comprising gene products.

The expression “coding mononucleotide microsatellites.” comprises repeat units of at least three equal mononucleotides A, T, G or C (n≧3), the repeat units being present in coding gene regions.

The term “coding dinucleotide microsatellites” comprises repeat units of at least three equal dinucleotides (AC, AG, AT, CA, CG, CT, GA, GC, GT, TA, TC, TG, n≧3), preferably at least six (n≧6) and more preferably at least nine (n≧9), the repeat units being located within coding gene regions.

The expression “genes having mutated cMNRs or cDNRs”, which can be isolated from MSI+ tumor cells, comprises such genes in full length as well as the mutations and parts thereof which contain the sequences coding for the neopeptides.

The expression “MSI+ tumor cells” comprises any tumor cells having a microsatellite instability. Such tumor cells may be available in any form, e.g. in a cell aggregation, in particular in a tumor, or be kept in culture as such. Preferred MSI+ tumor cells comprise the cell lines LoVo, KM12, HCT116, LS174 and SW48.

The expression “non-MSI+ (tumor) cells” comprises any cells having no microsatellite instability. Such cells may be of any kind and origin, e.g. the cells may be derived from healthy individuals or from tumors, and/or be tumor cell lines.

The terms mutations and “neopeptide-comprising gene products” point out that mutations are present in the coding microsatellites (cMNRs or cDNRs) of genes which can be isolated from MSI+ tumor cells as compared to the cMNRs or cDNRs of the corresponding genes from non-MSI+ (tumor) cells, the mutations being such that the genes code for neopeptide-comprising gene products. For example, the mutations are insertions and/or deletions of one or several mononucleotides and/or dinucleotides. The mutations result in reading frame shifts such that the gene products are present in the form of gene products comprising neopeptides, i.e. newly generated peptides.

In a preferred embodiment, the genes according to the invention are those differing from the genes, indicated in FIG. 1, of non-MSI+ (tumor) cells by mutations in the cMNRs or cDNRs and coding for neopeptide-comprising gene products. Specifically, the genes according to the invention have the mutations indicated in FIG. 2 in the cMNRs or cDNRs and code for the indicated neopeptide-comprising gene products.

Genes according to the invention can be identified and provided by various methods. It is favorable to use a method in which databases of non-MSI+ (tumor) cells are searched for gene sequences containing coding mononucleotide microsatellites (cMNRs) or dinucleotide microsatellites (cDNRs), these are used for detecting equal genes in MSI+ tumor cells and the latter genes are selected in such a way that they have mutations in the cMNRs or cDNRs as compared to the gene sequences from non-MSI+ (tumor) cells and code for neopeptide-comprising gene products. In order to detect the genes in the MSI+ tumor cell it is advantageous for the DNA thereof to be subjected to a PCR reaction with primers developed from the cMNR or cDNR-comprising gene sequences. The primers preferably comprise the sequences indicated in Table 1. As to the selection of the genes from the MSI+tumor cells it is also favorable to carry out the selection for those present in MSI+ tumor cells of different MSI+tumors of the same type with a frequency of about 1%-100%. In order to isolate the genes from the MSI+ tumor cells, it is also advantageous to use the corresponding gene sequences from the database for the preparation of appropriate primers and amplify with them the genes in the MSI+ tumor cells. Cloning of the amplified genes and the expressions thereof may then be carried out by common methods. For example, pGEMEX, pUC derivatives, pGEX-2T, pET3b and pQE-8 have to be mentioned as vectors for the expression in E. coli. For the expression in animal cells e.g. pKCR, pEFBOS, cDM8 and pCEV4 have to mentioned and the bacculovirus expression vector pAcSGH is NT-A is given by way of example for the expression in insect cells. The person skilled in the art is familiar with appropriate cells to express a genes present in the expression vectors. Examples of such cells comprise the E. coli strains HB101, DH 1, x1776, JM101, JM109, BL21 and SG13009, the yeast strain Saccharomyces cerevisiae and the animal cells L, NIH 3T3, FM3A, CHO, CO5, Vero and HeLa as well as the insect cells sf9. The person skilled in the art is also familiar with conditions of culturing transformed and/or transfected cells and isolating and purifying the expressed gene products. Reference is made to Sambrook et al., Molecular Cloning: A Laboratory Manual, 2^{nd} edition, Cold Spring Harbor Laboratory Press, Cold Spring Harbor, N.Y. (1989), by way of reference.

A further subject matter of the present invention relates to gene products which are encoded by the above genes. As to the genes according to the invention reference is made to the information given above. This information applies correspondingly to the gene products according to the invention. In particular, the gene products are those differing from the gene products of the genes indicated in FIG. 1 by mutations in the regions encoded by the cMNRs or cDNRs and comprising neopeptides. Especially the gene products comprise the mutations caused by cMNRs or cDNRs indicated in FIG. 2 and have the indicated neopeptides. Common methods can be used to provide the above gene products. Reference is made to the information given above. It may also be favorable to provide the neopeptides as such, in particular by means of peptide synthesis. Reference is made to Sambrook et al., supra, by way of supplement.

A further subject matter of the present invention relates to antibodies directed against the above gene products. Reference is made to the information given above as regards the gene products. This information applies correspondingly to the antibodies according to the invention. These antibodies are preferably monoclonal, polyclonal or synthetic antibodies or fragments thereof. In this connection, the term “fragment” refers to all parts of the monoclonal antibody (e.g. Fab, Fv or single chain Fv fragments) which have an epitope specificity the same as that of the complete antibody. A person skilled in the art is familiar with the production of such fragments. The antibodies according to the invention are preferably monoclonal antibodies. The antibodies according to the invention can be prepared in accordance with standard methods, the above gene products preferably serving as an immunogen. Methods of obtaining monoclonal antibodies are known to the person skilled in the art.

A further subject matter of the present invention also relates to kits which are suited for the study of MSI+tumors on a molecular level and for the diagnosis thereof. The kits can also be used to identify genes pertinent to MSI+ tumors. Such kits comprise one or several representatives of a gene, gene product, antibody and/or primer pair according to the invention. As regards genes, gene products and antibodies according to the invention reference is made to the information given above. The kits may also contain further substances, such as reverse transcriptase, DNA polymerase, ligase, buffer and reagents, e.g. labelings, dNTPs. In addition, the genes, gene products, antibodies and/or primer pairs according to the invention can be labeled. They may also be freely available or be immobilized by attachment to a solid carrier, e.g. a test tube, a microtration plate, a test rod, etc. The kits can also contain appropriate reagents for the detection of labelings or for labeling positive and negative controls, wash solution, dilution buffers, etc.

A further subject matter of the present invention relates to methods of immunizing an individual against MSI+ tumors and their preliminary stages, in which an individual is given an above gene in an expressible form or a gene product encoded by it. Reference is made to the above information on genes and gene products according to the invention.

For the administration of an above gene, the latter can be available as an RNA or DNA, preferably as a DNA. It may also be present as such, i.e. together with elements suited for its expression, or in combination with a vector. Examples of such elements are promoters and enhancers, such as CMV, SV40, RSV, metallothionein I and polyhedrin promoter and/or CMV and SV40 enhancer. Further sequences suited for expression follow from Goeddel: Gene Expression Technology: Methods in Enzymology 185, Academic Press, San Diego, CA (1990). Moreover, it is possible to use as vectors any vectors suited for expression in mammalian cells. These are e.g. pcDNA3, pMSX, pKCR, pEFBOS, cDM8 and pCEV4 as well as vectors derived from pcDNAI/amp, pcDNAI/neo, pRc/CMV, pSV2gpt, pSV2neo, pSV2-dhfr, pTk2, pRSVneo, pMSG, pSVT7, pko-neo and pHyg. Recombinant viruses, e.g. adenovirus, vaccinia virus or adeno-associated virus, can also be used as vectors.

For the administration of an above gene product, the latter may be present as such or in combination with carriers. It is favorable for the carriers not to have an immunogenic effect in an individual. Such carriers may be the individual's own or foreign proteins and/or fragments thereof. Carriers, such as serum albumin, fibrinogen or transferrin or a fragment thereof are preferred.

An individual who may be taken ill with an MSI+ tumor or is already ill therewith can be immunized using an above gene in an expressible form or a gene product encoded by it. Examples of such an individual are humans and animals as well as cells thereof. The immunization can be carried out under common conditions, the amount of gene to be administered or gene product encoded by it being easily determinable. It depends inter alia on whether the immunization of the individual rather focuses on an induction of antibodies directed against the gene product or on a stimulation of cytotoxic T cells directed against the gene product, e.g. CD8⁺ T cells. Both possibilities of immunization can be achieved by the present invention. Besides, the amount depends on whether the immunization aims at a prophylactic or therapeutic treatment. Moreover, the individual's age, sex and weight as well as further clinical parameters, e.g. kidney/liver function, play a role for determining the amount. It is favorable for the individual to be given by injection 100 μg-1 g of an above gene product or 10⁶-10¹² infectious particles of a recombinant virus containing an above expressible gene. The injection can be given intramuscularly, subcutaneously, intradermally or in any other form of application into several individual's sites. It may also be favorable to carry out one or several booster injections having approximately equal amount.

The present invention thus enables the detection of MSI+ tumors by means of diagnosis. These tumors can also be attacked by means of prophylaxis and therapy.

### BRIEF DESCRIPTION OF THE FIGURES

FIG. 1:

Genes having cMNRs or cDNRs from non-MSI+ (tumor) cells

FIG. 2:

Mutations in cMNRs or cDNRs from MSI+ tumors and neopeptides resulting therefrom. A comparison with corresponding genes from non-MSI+ (tumor) cells is also shown.

FIG. 3:

Study of mRNA expression in large intestine cancer cell lines using RT-PCR

The following examples explain the invention.

### EXAMPLE 1

### General Method

(A) Database Analyses

The EMBL database publication (EMBL Rel. 62, March 2000) was used as a basis for the search for mononucleotide and dinucleotide repeats in human coding sequences. A number of command routines and/or programs, what is called “Perl scripts” (Wall et al., Programming Perl. O'Reilly & Associates, Inc., (1996)) were written, which checked all human 109289-EMBL entries as regards the presence of coding sequences. These coding sequences were checked as regards the presence of mononucleotide and dinucleotide repeats having at least 6 bases (in the case of mononucleotide repeats) and 8 bases (in the case of dinucleotide repeats). In a database entry candidate, only the longest repeat of each nucleotide type was recorded as a mononucleotide repeat. As to the dinucleotide repeats, all of the 12 different kinds of dinucleotide repeats were considered. Entries for cDNA and genomic DNA were dealt with separately; if both DNA types were available for a gene, the genomic sequence was given priority. In addition, various filters were used. For example, entries shown to be pseudogenes were ruled out, since pseudogenes are usually not transcribed. Thus, such repeats are non-coding microsatellites. All of the identified candidate sequences were stored in a relational database for further analyses.

(B) Analysis of cMNR and cDNR Candidate Lists

In order to avoid the analysis of inappropriate candidate sequences, all candidates known to be pseudogenes or members of the immunoglobulin family were excluded. In addition, all candidates which had the repeats at the outermost 5′ or 3′ end of the known sequence as well as all CMNRs or cDNRs which on closer analysis of the primary data could be identified as cloning of sequencing artifacts were ruled out. All cMNRs having more than 9 A and T repeats and all C or G repeats having 9 repeat units were selected, since the probability of micrisatellite instability in tumors with the mutator phenotype increases with the number of repeat units (Strauss et al., Nucleic Acids Res. 25 (1997), 806-813). Two independent BLAST analyses were then carried out (Altschul et al., Nuc. Ac. Res. 25 (1997), 3389-3402). It was thus tried to identify homologous and genomic sequences to be thus able to identify the exon/intron transitions of the selected cDNAs, which was a confirmation for the fact that the repeat region in the cDNA did not form on account of a splicing process. In some cases, differences between the repeat sequences of the cDNAs and those of other published sequences were obtained in the very repeat region. Hence the genomic DNA had to be sequenced in each repeat region to verify the sequence information. Exon/intron transitions were identified by MALIGN analysis (HUSAR software program package) of a candidate cDNA and a homologous genomic DNA sequence.

(C) Cell Lines and Tumor Samples

14 human large intestine cancer cell lines were studied in each of the above genes as regards microsatellite changes. Five of the 14 large intestine cancer cell lines are classified as MSI+ (LoVo, KM12L4, HCT116, LS174T and SW48), while nine cell lines are classified as MSI-low or MSI-negative (CXF94, SW948, LS180, SW707, CaCo-2, HT29, Colo320DM, SW480 and CX-2). The cell lines SW48 and HCT 116 were obtained from ECACC [http://www.camr.org.uk/frame.htm]. The lines HT29, SW707, SW948, CaCo 2, CX-2, CXF94, SW480, COLO320DM, LoVo, LS174T and LS180 were obtained from the tumor bank of Deutsches Krebsforschungszentrum. KM12L4 cells were provided by Dr. I.J. Fidler, MD Anderson Cancer Center, Houston, U.S.A. 10 MSI+ CRC tumors, an MSI+ ovarioncus (B190 TU) and two MSI-low or MSI-negative CRC tumors (B215 TU and B245 TU2) were also analyzed. The paraffin-embedded tumors were taken from the archived material of Chirurgische Universitätsklinik Heidelberg or provided by Institut für Pathologie Mannheim. Genomic DNA of the tumor samples and the corresponding mucosa samples obtained by microdissection using standard methods were provided by Ch. Sutter (Sutter et al., Mol. Cell Probes, 13 (1999), 157-165). The MSI status was determined by means of the “NCI ICG-HNPCC”-microsatellite marker panel (Boland et al., Cancer Res. 58 (1998), 5248-5257) and additionally by means of amplification of the further microsatellite markers BAT40, ACTC, D13S153, D5S107 and D5S406.

(D) Genomic MSI Analyses

Primers were designed by means of the “PRIMER” program contained in the “HUSAR” program package and checked for further binding sites (sequence homologies) with respect to other human sequences by a “FASTA” analysis [HUSAR program package]. The primer positions were chosen such that they were as close as possible to the repeat region so as to obtain a short amplimer having a length of about 100 bp. This showed optimum results for an accurate fragment analysis of the DNA obtained from tissue samples embedded in paraffin. This also proved necessary for analyzing candidates having unknown genomic structure. All of the primers used are listed in below Table 1.

PCR reactions were carried out in a total volume of 25 μl (50 ng genomic DNA, 2.5 μl 10× reaction buffer (Life Technologies, Eggenstein-Leopoldshafen, Germany), 1.5 mM MgCl₂, 200 μM dNTPs, 0.25 μM of each primer and 0.5 U Taq DNA polymerase (Life Technologies). One primer was labeled with fluorescein at its 5′ end. After an initial denaturation step at 94° C. (4 min.), 35 cycles were carried out at 94° C. denaturation temperature for 30″, different attachment temperatures depending on the primer system at 57°-63° C. for 45″ and 72° C. extension temperature for 30″, this was followed by a final elongation step at 72° (6 min.). PCR products were analyzed on a 2% agarose gel. The amplification products were diluted 1:2 to 1:10 prior to the fragment analysis, and 1 μl of the dilute product was mixed with 5 μl application buffer (0.6% “blue dextran”, 100% formamide). The samples were denatured at 90° for 3 min., and then the fragments were separated by means of electrophoresis on an “ALF” DNA sequencing device (Amersham Pharmacia Biotech, Freiburg, Germany) using 6.6% polyacrylamide/7 M urea gels. The size, height and profile of the microsatellite peaks were analyzed by means of the “AlleleLinks” Software (Amersham Pharmacia Biotech).

TABLE 1

| | |
|---|---|
| PCR primers for genomic DNA | PCR primers for cDNA |

| | | | | | | | | |
|---|---|---|---|---|---|---|---|---|
| | | SEQ | | SEQ | | SEQ | | SEQ |
| Gene | sense 5′-3′ | ID NO. | antisense 5′-3′ | ID NO. | sense 5′-3′ | ID NO. | antisense 5′-3′ | ID NO. |

| | | | | | | | | |
|---|---|---|---|---|---|---|---|---|
| MNRs | | | | | | | | |
| FLT3LG | GGG ATG ACG TGG | 1 | GTG ATC CAG GGC | 2 | CCT ATC TCC TCC | 3 | GTG ATC CAG GGC | 4 |
| | TGG TG | | TTC AGC | | TGC TGC TG | | TTC AGC | |
| SYCP1 | CCC CTT CAT CTC | 5 | CAC TGA TTC TCT | 6 | CAG TGA AGA CAC | 7 | CAC TGA TTC TCT | 8 |
| | TAA CAA CCC | | GAA ATT AAA CAA | | CAA CAA AAC C | | GAA ATT AAA CAA | |
| | | | ATA AC | | | | ATA AC | |
| SLC4A3 | TGG AGT GGA TGA | 9 | CTT CTG TGG GGT | 10 | TGG AGT GGA TGA | 11 | ATC TGT GGG CAC | 12 |
| | GGA AGA GG | | CCC TGA G | | GGA AGA GG | | CTG CTG | |
| aC1 | CCA GAA GCA AAT | 13 | TTT TGC GTG TTC | 14 | CCA GAA GCA AAT | 15 | CAC CCT CTC TCT | 16 |
| | TCA CAA GAC | | CTT CCT TC | | TCA CAA GAC | | TCT CCA GTA TTC | |
| PTHL3 | TTT CAC TTT CAG | 17 | GAA GTA ACA GGG | 18 | GGA AAC TAA CAA | 19 | GAA GTA ACA GGG | 20 |
| | TAC AGC ACT TCT | | GAC TCT TAA ATA | | GGT GGA GAC G | | GAC TCT TAA ATA | |
| | G | | ATG | | | | ATG | |
| SLC23A1 | GAC TAC TAC GCC | 21 | TGT TTA TTG CGT | 22 | AAA GGA TGG ACT | 23 | AAG GAC GAG CCC | 24 |
| | TGT GCA CG | | GGA TGG G | | GCG TAC AAG | | AAA GAA G | |
| GART | AGT GTT GAA GAA | 25 | TGT TCC AGA TAT | 26 | GAA CAT CCC CAG | 27 | TGT TCC AGA TAT | 28 |
| | TGG CTC CC | | TAA GAC AGC CAC | | AGT CCT CC | | TAA GAC AGC CAC | |
| MAC30X | TGT TGC GGA GCC | 29 | AAC CAC CCT GTA | 30 | CCT GGT TTA AGT | 31 | AAC CAC CCT GTA | 32 |
| | CCT AC | | GGC ATC TC | | CCT TTC TGT TTT | | GGC ATC TC | |
| PRKDC | GAC TCA TGG ATG | 33 | TTT GAA AAT AAC | 34 | CAG CCC TGG ACC | 35 | GAC AAC CCC TTC | 36 |
| | AAT TTA AAA TTG | | ATG TAA ATG CAT | | TTC TTA TTA A | | AGA CAT CC | |
| | G | | CTC | | | | | |
| ATR | TCT TCT GTA GGA | 37 | TGA AAG CAA GTT | 38 | AGC TCC CAT GAA | 39 | TGA AAG CAA GTT | 40 |
| | ACT TCA AAG CC | | TTA CTG GAC TAG | | GTA ATC CG | | TTA CTG GAC TAG | |
| | | | G | | | | G | |
| MBD4 | TGA CCA GTG AAG | 41 | GTT TAT GAT GCC | 42 | TGA CCA GTG AAG | 43 | GTC GTG GGG GGC | 44 |
| | AAA ACA GDC | | AGA AGT TTT TTG | | AAA ACA GCC | | TAA GAG | |
| SEC63 | AGT AAA GGA CCC | 45 | TGC TTT TGT TTC | 46 | TGA AAA GGA GCA | 47 | TGC TTT TGT TTC | 48 |
| | AAG AAA ACT GC | | TGT TGC TTT G | | GTC CAT CTG | | TGT TGC TTT G | |
| OGT | TCA CTT TTG GCT | 49 | GGG AGG GAA AGG | 50 | TCA CTT TTG GCT | 51 | TGT CAA AAA TGC | 52 |
| | GGT CAG AG | | AGG TAA AG | | GGT CAG AG | | GTG CCT C | |
| HPDMPK | GCT TGA TCC TGT | 53 | CTG AAT GGA GAA | 54 | TCC TAC TGG ATG | 55 | CTG AAT GGA GAA | 56 |
| | TGA TTT TCT ACT | | GAA AGT GAG ATG | | TGC TGC C | | GAA AGT GAG ATG | |
| | C | | | | | | | |
| U79260 | TTT GTT ATA TCC | 57 | AGC CTG GTG ACA | 58 | CAT TAA GCA AAG | 59 | AGC CTG GTG ACA | 60 |
| | CAT TAG GTG CC | | GAG TGA GAC | | CAG CCA GG | | GAG TGA GAC | |
| DNRs | | | | | | | | |
| K1AA0040 | CAT CTC AAT ATG | 61 | CTT GCC CAC GTA | 62 | CAA GAA GTA AGG | 63 | GTG CAT TAT TTC | 64 |
| | GTT CCC AAG TG | | CCT GCT AC | | TGG AAG GAG G | | AGG GGT TCC | |

(E) Confirmation of the Sequence

All of the coding microsatellites were confirmed by “thermocycle” sequencing. PCR reactions were carried out as described above. PCR products were purified by means of the “QIAquick” PR purification kit (Qiagen, Hilden, Germany) and sequenced with the corresponding primers using the “Big Dye terminator cycle sequencing kit (Perkin Elmer, Darmstadt, Germany).

(F) Expression Analyses and cDNA-MSI Analyses

Poly(A)RNA of 14 large intestine cancer cell lines were extracted by means of the oligo(dT) cellulose method (Vennstrom and Bishop, Cell 28 (1982), 135-143). The quality of the RNA preparation and the reverse transcription were checked by means of GAPDH amplification (Hsu et al., Int. J. Cancer, 55: 397-401, 1993). Primer pairs permitting a differentiation according to size between cDNA and genomic DNA amplimers possibly contained as a contamination, were considered appropriate for the expression analysis by semi-quantitative RT-PCR. In the case of an unknown exon structure, primers were designed which were localized on the cDNA, and it was checked whether genomic PCR yielded either an amplification product the same as that of the RT-PCR, a longer one or none at all. All of the primers used are listed in Table 1. 100 ng poly(A+)RNA were subjected to reverse transcription by means of 0.5 μg oligo(dT)₁₂₋₁₈ in a final volume of 20 μl with 200 units M-MLV Reverse Transcriptase (SuperScript, Life Technologies) at 37° C. for 1 hour. In order to check the RNA integrity and the synthesis of the first cDNA strand, control PCR reactions were carried out by means of GAPDH-specific primers (Hsu et al., Int. J. Cancer 55 (1993), 397-401). PCR reactions were carried out in a total volume of 50 μl (1 μl cDNA, 5 μl 10× reaction buffer (Life Technologies), 1.5 mM MgCl₂, 200 μM dNTPs, 0.25 μM of each primer and 0.5 units Taq DNA polymerase (Life Technologies) by means of the above amplification protocol described above for the amplification of genomic DNA. The PCR products were separated on 2% agarose gels and made visible on ethidium bromide staining.

PCR reactions for cDNA-MSI analyses were carried out as described for the expression analyses, except that a primer labeled with fluorescein at its 5′ end was used. The fragment analysis was carried out as described for the genomic analysis.

### EXAMPLE 2

### Computer-Assisted Analyses

The computer database sequence analysis resulted in 365 candidates for mononucleotide repeats having a minimum length of 9 bases (total: 17654 mononucleotide repeats having a length of ≧6 bases). In addition, 2028 dinucleotide repeats having a minimum length of 8 bases were found. The longest mononucleotide region comprised 32 bases and the longest dinucleotide region comprised 42 bases, i.e. 21 repeat units.

### EXAMPLE 3

### Identification of cMNR

All cMNR having 10 or more repeat units and all C or G repeat regions having 9 or more repeat units were analyzed for this purpose, since it was assumed that the mutation rate was increased in the relatively long repeat regions. Moreover, all candidate sequences which satisfied further exclusion criteria were ruled out from an analysis. A total of 43 cMNR candidate sequences were thus obtained, which comprised 12 duplicates so that 31 different candidate sequences were selected.

These candidate sequences had to be verified experimentally as microsatellites in coding regions. Therefore, primers flanking the repeat regions had to be designed for each candidate sequence. The full information about the genomic structure could be obtained by means of sequence comparison of the cDNAs with databases for the genomic sequence. The systematic sequence comparison supplied information about exon/intron transitions and coding regions for 9 of the 31 candidate sequences.

Therefore, primer pairs on the basis of the cDNA sequence had to be designed for the other 22 cDNA sequences. The amplification of the repeat regions in both the cDNA and the corresponding genomic DNA resulted in identical PCR products in further 9 of the 22 candidates, which shows that these 9 sequences contain the mononucleotide region in a coding region. The PCR reaction of the genomic DNA sequences was negative in 13 mononucleotide regions or resulted in amplimers longer than those obtained by means of the amplification of the corresponding cDNA. Another analysis of the genomic structure of the corresponding gene was thus required for each of these candidate sequences. A total of 18 mononucleotide regions were subjected to a sequence analysis.

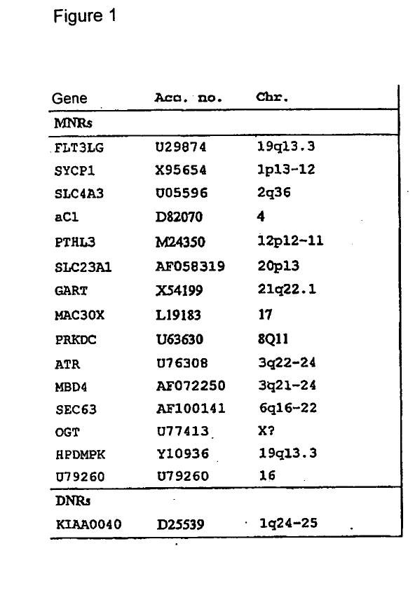

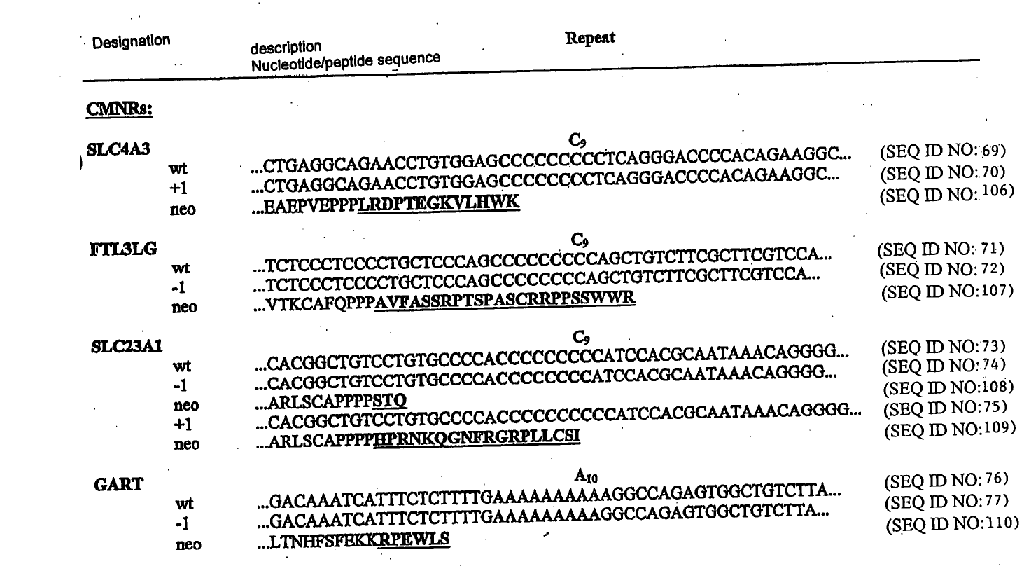

It was possible to confirm repeat regions by sequence analyses in 17 of the 18 cases. Only one candidate did not show the expected A₁₄ repeat. With two candidate sequences, the repeat region was located within the predicted coding sequence of the genomic DNA sequences, yet it was not possible to show an expression by means of RT-PCR analyses. Moreover, no information could be identified as regards ESTs or a partial coding sequence homologous to the repeat region. These studies were carried out by means of the “EST Clustering software” (Husar program package). Thus, the sequence analysis together with the confirming experiments formed the basis for the identification of 15 cMNRs (cf. FIG. 1).

### EXAMPLE 4

### Detection of cDNR

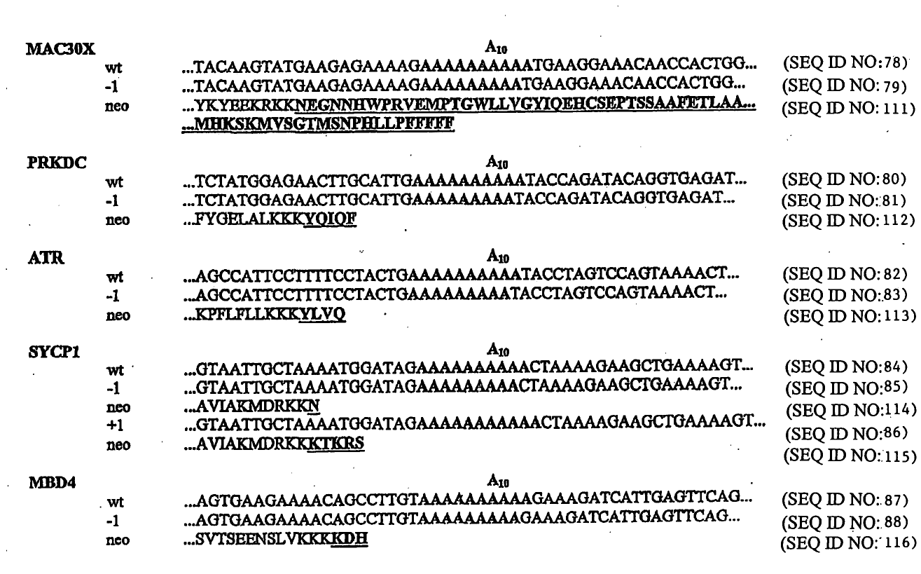

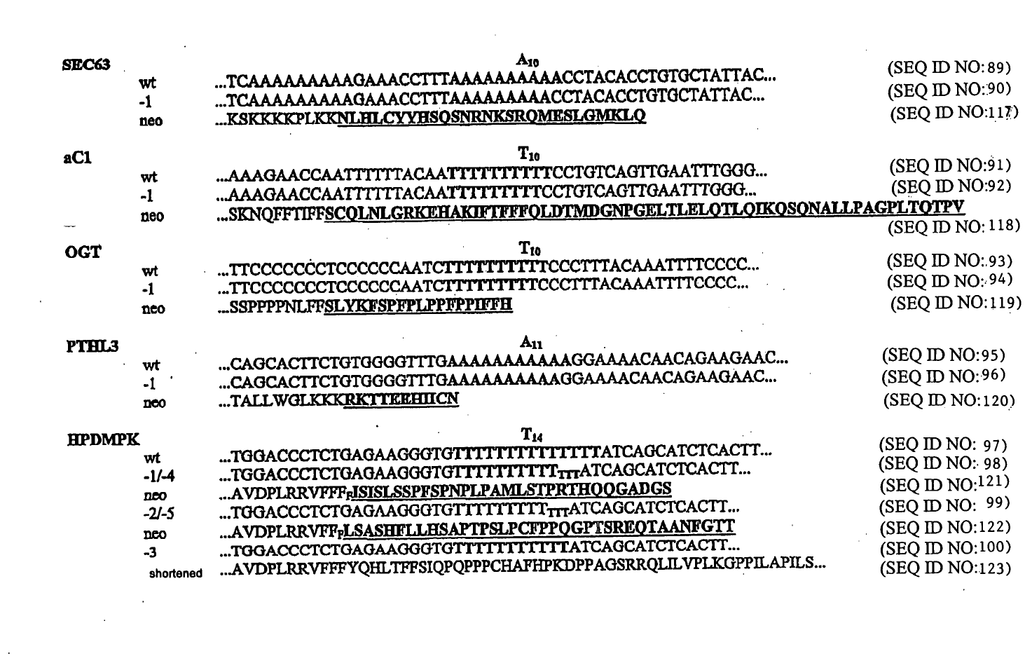

The same strategies as used for the sequence analysis and the experimental confirmation of the sequence data were applied to the cDNRs, the longest cDNR candidates having been started with. Having identified cDNA sequences of 4 cDNR candidates, the MSI status in MSI+ an MSI large intestine cancer cell lines and MSI+ tumors was analyzed. One of these candidates ((AC)₇) showed a mutation in an MSI+tumor, but no mutation in the non-MSI+ (tumor) cells (cf. FIG. 2). It is assumed that the mutation rate is increased in the case of cDNRs having a repeat number of above 9.

### EXAMPLE 5

### Studies as to the Mutation Rates of the cMNRs

In particular, varying mutation rates ranging from 40 to 80% were observed in the three C₉-cMNRs and rates ranging from 10 to 100% were detected in the A₁₀-cMNRs, whereas the T₁₀ and N_{≧11}-cMNRs showed constantly higher mutation rates between 75 and 100%. In three cMNR markers ((SYCP1 (A10), ATR (A10), and MBD4 (A10)) only minor mutation frequencies could be identified in MSI+ cell lines and tumor samples. In MSI+ cell lines and MSI+ tumors, however, high mutation rates were detected for the two cMNR markers (HPDMPK (T14) and U79260 (T14)): All of the 5 MSI+ cell lines and 10 of 11 MSI+ tumors showed a change in sequence as regards HPDMPK. Analogous results were found for the mono-cMNRs in the U79 260 gene which was mutated in all of the 5 MSI+ cell lines and in 9 of 11 MSI+ tumors (cf. below Table 2).

TABLE 2
Frequency of mutations in cMNR in MSI+ tumor cell lines and MSI+ tumors

| Gen | Repeat | LoVo | KM12 | HCT116 | LS174T | SW48 | T1 | T2 | T3 | T4 | T5 | T6 | T7 | T8 | T9 | T10 | [symbol] |
|---|---|---|---|---|---|---|---|---|---|---|---|---|---|---|---|---|---|
| FLT3LG | C₉ | • | • | ∘ | ∘ | • | ∘ | ∘ | • | • | ∘ | • | • | ∘ | ∘ | | |
| SYCP1 | A₁₀ | • | ∘ | ∘ | ∘ | ∘ | • | ∘ | ∘ | ∘ | • | ∘ | ∘ | ∘ | ∘ | ∘ | |
| SLC4A3 | C₉ | ∘ | • | ∘ | • | ∘ | ∘ | ∘ | • | ∘ | ◯ | ∘ | ∘ | ∘ | ∘ | • | |
| aC1 | T₁₀ | • | ∘ | ∘ | • | • | • | • | • | • | ◯ | • | • | ∘ | ∘ | • | |
| PTHL3 | A₁₁ | • | • | • | • | • | • | • | • | • | • | ∘ | • | • | • | • | |
| SLC23A1 | C₉ | ∘ | • | • | • | • | • | • | ∘ | ∘ | • | ∘ | • | • | • | ∘ | |
| GART | A₁₀ | ∘ | • | ∘ | ∘ | • | ∘ | • | ∘ | ∘ | ◯ | ∘ | ∘ | • | • | ∘ | |
| MAC30X | A₁₀ | ∘ | ∘ | • | ∘ | ∘ | ∘ | • | ∘ | ∘ | ◯ | ∘ | • | ∘ | ∘ | ∘ | |
| PRKDC | A₁₀ | ∘ | • | ∘ | ∘ | ∘ | • | • | ∘ | ∘ | ◯ | ∘ | ∘ | • | • | ∘ | |
| ATR | A₁₀ | ∘ | ∘ | ∘ | ∘ | ∘ | • | ∘ | ∘ | ∘ | | ∘ | ∘ | ∘ | ∘ | • | |
| MBD4 | A₁₀ | ∘ | ∘ | • | ∘ | ∘ | ∘ | ∘ | ∘ | ∘ | ◯ | ∘ | ∘ | ∘ | ∘ | ∘ | |
| SEC63 | A₁₀ | • | • | • | • | • | ∘ | • | • | • | • | ∘ | • | • | • | • | |
| OGT | T₁₀ | • | • | ∘ | • | ∘ | ∘ | ∘ | ∘ | • | • | ∘ | • | • | • | ∘ | |
| HPDMPK | T₁₄ | • | • | • | • | • | • | • | • | • | • | ∘ | • | • | • | • | |
| U79260 | T₁₄ | • | • | • | • | • | • | ∘ | • | • | • | ∘ | • | • | • | • | |

∘ Mutation non available
• mutation available

### EXAMPLE 6

### Expression Analyses of cMNRs

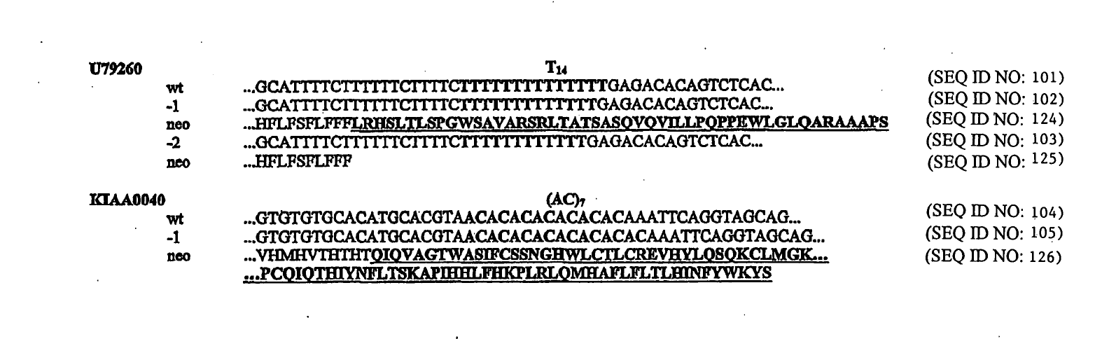

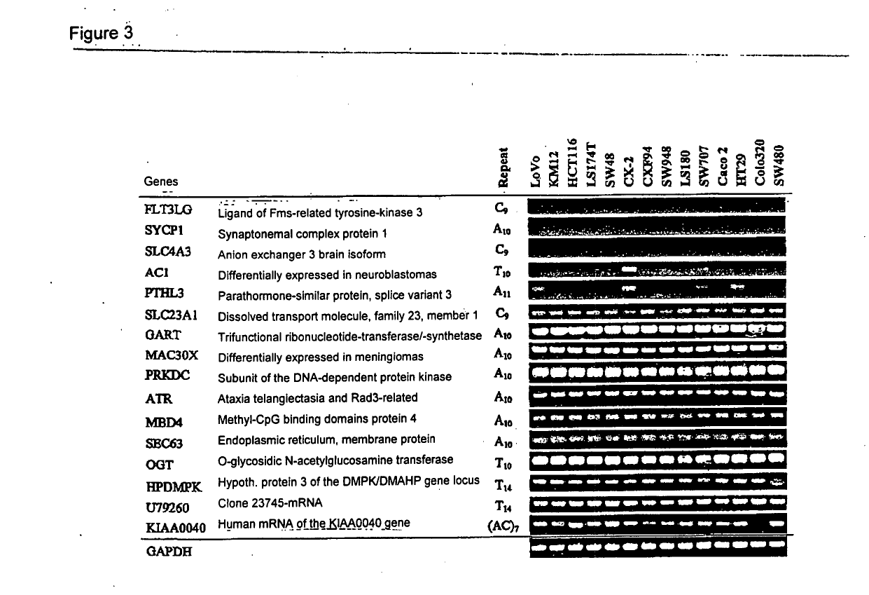

The expression levels of the above 15 cMNR-containing genes differed widely and varied between non-detectable expression and constantly strong transcription activity in all of the 14 tested large intestine cancer cell lines. The SYCP1 gene involved in meiosis and the gene coding for the hematopoietic growth factor FLT3LG were not expressed in large intestine cancer cell lines. The gene HPDMPK located downstream of the gene locus for two genes associated with myotonic dystrophy (Dystrophia myotonica) and coding for a hypothetical protein and the gene coding for the ER membrane protein SEC63 were not expressed very highly, yet were expressed constantly, in all of the cell lines. The aC1-mRNA and splice variant 3 of the PTHrP gene (PTHL3) were expressed in large intestine cancer cell lines to a different extent. Both genes were expressed in about 50% of the investigated cell lines. The GART gene coding for the trifunctional ribonucleotide synthetase, the PRKDC gene coding for the DNA-dependent protein kinase and the ATR gene connected with the cell cycle were highly expressed in large intestine cell lines. MAC30X is also highly expressed in large intestine cancer cell lines (cf. FIG. 3). In summary, it can be pointed out that the expression levels of the corresponding genes do not correlate with the MSI status of the affected cell lines.

### EXAMPLE 7

### MSI Analysis of cDNAs

A fragment analysis of amplified cDNAs of the above cMNRs of 14 large intestine cancer cells lines was carried but: Three cMNRs showed the wild-type cDNA in affected cell lines (GART) or in most affected cell lines (SEC63). In seven cMRNs there was correspondence between the genomic and transcribed sequences (MAC30X, HPDMPK, U79260, MBD4 and ATR).

### EXAMPLE 8

### Stimulation of CD8⁺ T Cells Against an Above Gene Product and Lysis of MSI+ Tumor Cells Expressing this Gene Product

(a) Stimulation of CD8* T Cells Against a Neopeptide According to the Invention

Peripheral blood lymphocytes (PBL) from an HLA-A0201-positive healthy proband were purified by density centrifugation on a Ficoll Paque® gradient. T lymphocytes were obtained by separating the B lymphocytes and/or the monocytes using antibody-linked magnetobeads (CD11, CD16, CD19, CD36, and CD56) (Pan T-cell isolation Kit®, Milteny, Bergisch Gladbach, Germany). About 2×10⁷ T cells were obtained from 30 ml blood. Of these about 2×10⁶ T cells were stimulated with autologous cells B cells activated on CD40 (about 5×10⁵) which had been loaded with one of the HLA-A0201-restricted neopeptides of below Table 3 (cf. also FIG. 2), i.e. cocultured in 24 well plates. This stimulation was repeated weakly for a period of five to six weeks.

TABLE 3
Examples of HLA-A0201-restricted neopeptides
encoded by mutated cMNR

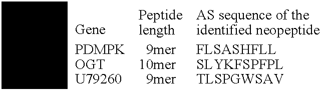

By means of known IFN-gamma ELISpotanalysis, the reactivity over the neopeptides was determined weakly, starting on day 0. On day 28, a reactivity of 1760 specific cells/1,000,000 cells against peptide #16 (SLYKFSPFPL) was observed, on day 35 there was found one of 1123 specific cells/1,000,000 cells against peptide #15 (FLSASHFLL) and one of 733 specific cells/1,000,000 cells against peptide #21 (TLSPGWSAV). The strength of the reaction was thus within ranges usually only achieved by means of viral antigens, the value for the GILGFVFTL peptide, which was derived from a matrix protein of the influenzavirus, was 1170 specific cells/1,000,000 on day 35. Hence it becomes obvious that it is possible to stimulate activated CD8⁺ T cells against the neopeptides according to the invention.

(b) Lysis of Cells Loaded with Neopeptides According to the Invention

After another restimulation, the cytotoxic potential of the activated CD8⁺ T cells was tested for the neopeptide-loaded HLA-A2.1⁺ colon carcinoma cell lines SW480 and HCT 116 as well as T2 cells. Unloaded cells served as a control. 1×16⁶ cells each were labeled radioactively using ⁵¹Cr (100 μCi) at 37° C. for 1 h and cocultured with increasing amounts of activated CD8⁺ T cells for 4 h. The specific lysis of the respective cell line was determined by measuring the released radioactivity in the supernatant. It turned out that the HLA-A0201-expressing cell lines can be lyzed when they are loaded with neopeptides, unloaded cells are not lyzed.

Furthermore, competition experiments were carried out. It was possible to compete with the release of radioactivity and thus the specific cytotoxic activity of the T cells by the addition of an excess (50 “cold” to 1 “hot” neopeptide-loaded cell) of neopeptide-loaded T2 cells which are, however, not labeled radioactively to a reaction batch with radioactively labeled neopeptide-loaded T2 cells and activated CD8⁺ T cells. Thus, it becomes evident that the CD8⁺ T cells directed against the neopeptides specifically detect and lyze the neopeptide-epxressing tumor cells.

1-13. (cancelled).

14. A gene encoding one or more mononucleotide microsatellites (cMNR) or dinucleotide microsatellites (cDNR), wherein the gene is isolated from a MSI+ tumor cell and differs from the corresponding gene from a non-MSI+(tumor) cell by a mutation in the cMNR or cDNR and codes for a gene product which comprises a neopeptide, wherein the corresponding gene from a non-MSI+ (tumor) cell is one selected from the group consisting of FLT3LG, SYCP1, SLC4A3, aC1, PTHL3, SLC23A1, GART, MAC30X, PRKDC, ATR, MBD4, SEC63, OGT, HPDMPK, U79260 and KIAA0040.

15. The gene according to claim 14, wherein the gene isolated from a MSI+ tumor cell has a mutation selected from the group consisting of SEQ ID NOs: 70, 72, 74, 75, 77, 79, 81, 83, 85, 86, 88, 90, 92, 94, 96, 98, 99, 100, 102, 103, and 105.

16. A gene product encoded by the gene according to claim 14.

17. A gene product encoded by the gene according to claim 15.

18. An antibody directed against the gene product according to claim 16.

19. An antibody directed against the gene product according to claim 17.

20. A method for identifying the gene according to claim 14 or 15, comprising: searching a database of non-MSI (tumor) cells for cMNR- or cDNR-containing gene sequences, and identifying a cMNR- or cDNR-containing gene sequence in a MSI+ tumor cell with the cMNR- or cDNR-containing gene sequences obtained from the searching step, wherein the cMNR- or cDNR-containing gene sequence identified contains one or more mutations compared to the corresponding gene in a non-MSI+(tumor) cell, wherein the cMNR- or cDNR-containing gene sequence codes for a gene product which comprises a neopeptide.

21. The method according to claim 20, wherein said identifying comprises carrying out a PCR reaction on the non-MSI+ (tumor) cell with primers developed from the cMNR- or cDNR-comprising gene sequences obtained from the searching step.

22. The method according to claim 20, wherein the primers are selected from the group consisting of SEQ ID NOs: 1-60.

23. The method according to claim 20, wherein the cMNR- or cDNR-containing gene sequence identified from the MSI+ tumor cells are present in MSI+ tumor cells of various MSI+ tumors of equal type with a frequency of 1%-100%.

24. A kit comprising one or more genes according to claim 14 or 15, one or more gene products according to claim 16 or 17, one or more antibodies according to claim 18 or 19, one or more primer pairs selected from the group consisting of SEQ ID NOs: 1-60, or a combination thereof.

25. A method for molecular investigation of MSI+ tumors and their preliminary stages, or the diagnosis thereof, comprising: detecting MSI+ tumors and their preliminary stages with the gene according to claim 14 or 15, or the gene products according to claim 16 or 17, or the antibodies according to claim 18 or 19.

26. A method for molecular investigation of MSI+ tumors and their preliminary stages, or the diagnosis thereof, comprising: detecting MSI+ tumors and their preliminary stages with the kit according to claim 22.

27. A method for immunizing an individual against MSI+ tumors and their preliminary stages comprising: immunizing said individual with a gene according to claim 14 or 15, wherein the gene is expressible in the individual, or a gene product according to claim 16 or 17.

28. The method according to claim 27, wherein the immunizing results in a prophylactic or therapeutic treatment for MSI+ tumors.
